(19) United States
(12) Patent Application Publication
(10) Pub. No.: US 2022/0370772 A1
(43) Pub. Date: Nov. 24, 2022

(54) DISPENSER DEVICE FOR DELIVERY OF COMPOSITIONS INTO VAGINAL OR ANAL ORIFICES

(71) Applicant: Record Buck Farms Medical Cannabis LLC, Howey In The Hills, FL (US)

(72) Inventor: John V. Rivera, Howey In The Hills, FL (US)

(73) Assignee: Record Buck Farms Medical Cannabis LLC, Howey In The Hills, FL (US)

(21) Appl. No.: 17/328,413

(22) Filed: May 24, 2021

(51) Int. Cl. A61M 31/00

(52) U.S. Cl. CPC A61M 31/00; A61M 2202/04; A61M 2205/075; A61M 2210/1067; A61M 2210/1475

(57) ABSTRACT

A dispenser device for delivering a composition into an anal or vaginal orifice includes an applicator having a shaft, one or more channels, and a plurality release ports. The shaft may extend from a tip to a base and have an outer surface. The plurality of release ports may be positioned along the outer surface of the shaft, and the one or more channels may extend through the shaft and be in fluid communication with the plurality of release ports.

### TECHNICAL FIELD

The present disclosure is directed to a dispenser device for dispensing a composition. More specifically, the preset disclosure is directed to a dispenser device for dispensing a composition, such as a medication, lubricant, or cleaning composition, within a vaginal or anal orifice.

### BACKGROUND

The anus and vagina are susceptible to pain, inflammation, dryness, and infections. These orifices also provide locations for quick transmucosal absorption of medication. Anal and vaginal suppositories are used to administer medication at anal and vaginal orifices. These suppositories typically breakdown or otherwise release their contents within the orifice for transmucosal administration. Medicated creams or ointments may also be used to combat local infections and may be applied to the vagina or anus. However, such applications may be cumbersome and inexact. For example, it may be difficult to evenly administer a desired amount of a treatment composition or to apply a desired amount of treatment composition at deep locations. Further, the need for frequent reapplications in some instances leads to low drug compliance in some patients. Sexual lubricants or moisturizers may be used to lubricate sex toys or sexual organs, such as the vagina or anus. Administration of sexual lubricants suffers from many of the same difficulties as those for creams and ointments. For example, the duration of lubrication may be limited and frequent reapplication may be required.

What is needed are improved dispensing devices and methods to provide medications, lubrication, cleaning, or other compositions to anal or vaginal orifices.

### SUMMARY

In one aspect, a dispenser device for delivering a composition into an anal or vaginal orifice includes an applicator for dispensing the composition. The applicator may include a shaft, one or more channels, and a plurality release ports. The shaft may extend from a tip to a base and have an outer surface. The plurality of release ports may be positioned along the outer surface of the shaft, and the one or more channels may extend through the shaft and be in fluid communication with the plurality of release ports.

In one embodiment, the device further includes a reservoir configured to contain the composition to be delivered from the device. The reservoir may be configured to couple to the applicator and being in fluid communication with the one or more channels when so coupled. The reservoir and applicator may each include a fitting configured to engage and couple the reservoir to the applicator.

In one example, the device further comprises a handle that is integrated with the reservoir.

In another or a further example, the reservoir includes a flexible and/or elastomeric material configured to be squeezed by a user to urge a composition within the reservoir to flow from the reservoir and through the one or more channels.

In another or a further example, the applicator fitting is positioned adjacent to the base of the shaft and includes a grooved female connector and the reservoir fitting includes a grooved male connector configured to be positioned within the female connector and rotated to couple the applicator and the reservoir.

In an above or another embodiment, the one or more channels comprise a bulk channel that extends from the base of the shaft toward the tip of the shaft. The one or more channels may further include a plurality of branch channels. Each branch channel may extend between the bulk channel and one of the release ports.

In an above or another embodiment, the plurality of release ports are located along the tip and sides of the shaft.

In an above or another embodiment, the plurality of release ports include protrusions that protrude from adjacent portions of the outer surface of the shaft.

In another aspect, a method of administering a composition to a subject includes inserting a dispensing device within an anal or vaginal orifice and ejecting the composition from a plurality of release ports within the anal or vaginal orifice. The dispensing device may include an applicator including a shaft that extends from a tip to a base and including an outer surface. A plurality of release ports may be positioned along the outer surface of the shaft. One or more channels may extend to and be in fluid communication with the plurality of release ports. The one or more channels may extend through the shaft from the plurality of release ports toward the base of the shaft. A reservoir configured to contain the composition to be dispensed from the device may be in fluid communication with the one or more channels.

In one embodiment, the reservoir comprises a flexible and/or elastomeric material that is squeezed to urge the composition within the reservoir to flow from the reservoir and through the one or more channels and out of the plurality of release ports.

In the above or another embodiment, the reservoir and applicator each include a fitting configured to engage and couple the reservoir to the applicator. In one example, the applicator fitting is positioned adjacent to the base of the shaft and includes a grooved female connector and the reservoir fitting includes a grooved male connector configured to be positioned within the female connector and rotated to couple the applicator and the reservoir.

In an above or another embodiment, the one or more channels include a bulk channel that extends from the base of the shaft toward the tip of the shaft. In one example, the one or more channels further include a plurality of branch channels, each of which extend between the bulk channel and one of the release ports.

In an above or another embodiment, the plurality of release ports are located along the tip and sides of the shaft.

In an above or another embodiment, the plurality of release ports include protrusions that protrude from adjacent portions of the outer surface of the shaft.

In any of the above or another embodiment, the composition comprises one or more cannabinoids. In one example, the composition is a liquid, cream, or an oily blend.

### BRIEF DESCRIPTION OF THE DRAWINGS

The novel features of the described embodiments are set forth with particularity in the appended claims. The described embodiments, however, both as to organization and manner of operation, may be best understood by reference to the following description, taken in conjunction with the accompanying drawings in which:

FIG. 1 is a side view of a dispenser device according to various embodiments described herein;

FIG. 2 is a top view of a dispenser device according to various embodiments described herein;

FIG. 3 is a bottom view of a dispenser device according to various embodiments described herein;

FIG. 4 is a front end view in cross-section of a dispenser device according to various embodiments described herein; and

FIG. 5 is a reservoir view of a dispenser device according to various embodiments described herein.

### DESCRIPTION

The present disclosure describes a dispenser device configured to dispense a composition. Various embodiments of the dispenser device may be designed, e.g., sized and shaped, to massage the prostrate, anal canal, and/or vaginal canal while being capable of delivering a composition, which may include a set of compositions comprising one or more treatments for a health condition. For example, the dispenser device may be configured to dispense a composition within an anal or vaginal orifice. The dispenser device may similarly be configured to dispense a composition into other body orifices or onto body surfaces. In one embodiment, the dispenser device may be configured to dispense a composition into or onto equipment. The composition may include any suitable composition and/or components thereof, such as those described herein. For example, the composition may include one or more lubricants, cleaners, or pharmaceutical components such as one or more antifungals, antibiotics, nonsteroidal anti-inflammatory, analgesics, or cannabinoids.

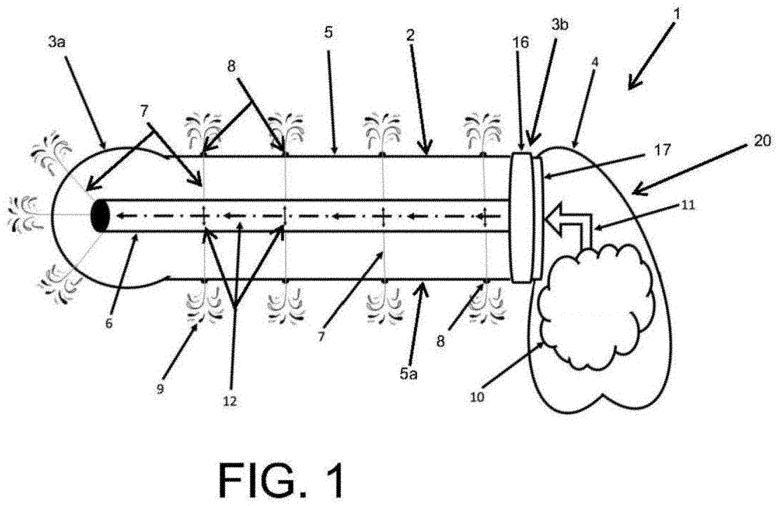

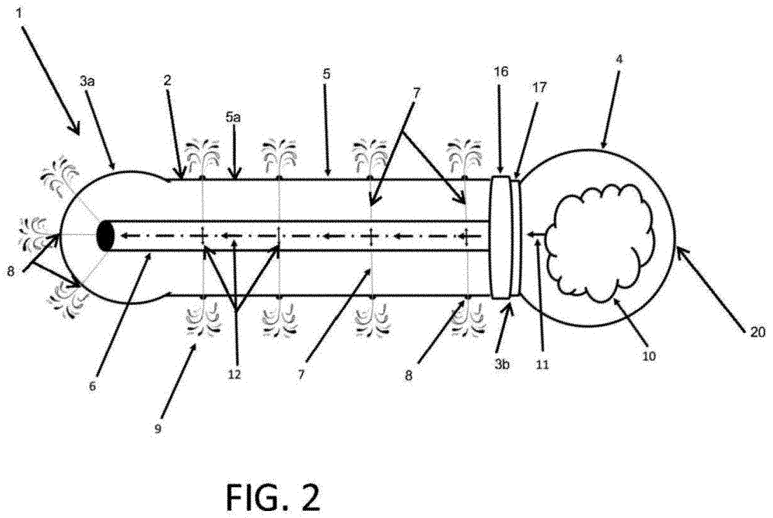

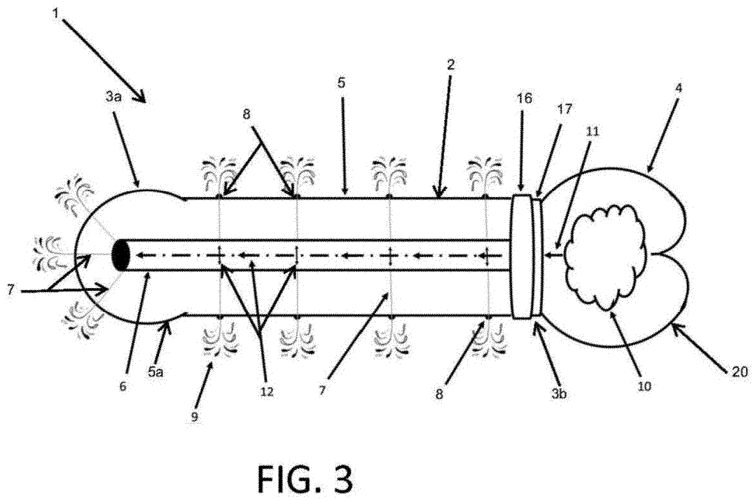

With reference to FIGS. 1-5, illustrating various features of a dispenser device 1 according to various embodiments described herein, wherein like features are identified by like numbers, the dispenser device 1 may include an applicator 2 through which a composition 10 may be flowed 11, 12 and thereafter released 9 from the applicator 2 and multiple locations. A handle 20 may be provided to allow a user to manipulate the applicator 2.

The applicator 2 may include a shaft 5 that extends between a tip 3a and a base 3b and that includes an outer surface 5a. The applicator 2 may be innervated with one or more channels 6, 7 that extend through the shaft 5 from a reservoir 4 to a plurality of release ports 8 positioned along the outer surface 5a of the shaft 5. In use, a composition 10 may be flowed 11, 12 along the one or more channels 6, 7 for release from the applicator 2 at the release ports 8 at the outer surface 5a.

In the illustrated embodiment, the applicator 2 includes a shaft 5 that extends to a tip 3a wherein the outer surface 5a includes release ports 8 along the tip 3a as well as sides of the shaft 5. Each release port 8 is connected to a branch channel 7 that fluidically couples to a bulk channel 6 that is further fluidically coupled to the reservoir 4. A bulk channel 6 refers to a channel through which composition may flow from the reservoir 4 to two or more release ports 8. A branch channel 7 fluidically couples between a bulk channel 6 and a release port 8. However, in some embodiments, a flow path between a bulk channel 6 and a release port 8 may include multiple branch channels 7. In the illustrated embodiment, a bulk channel 6 extends along a length of the shaft 5 and connects to a plurality of branch channels 6 that extend laterally from the bulk channel 6 in multiple directions, each to a release port 8 located along the outer surface 5a. In some embodiments, release ports 8 may be provided along only the sides of the shaft 5 or only the tip 3.

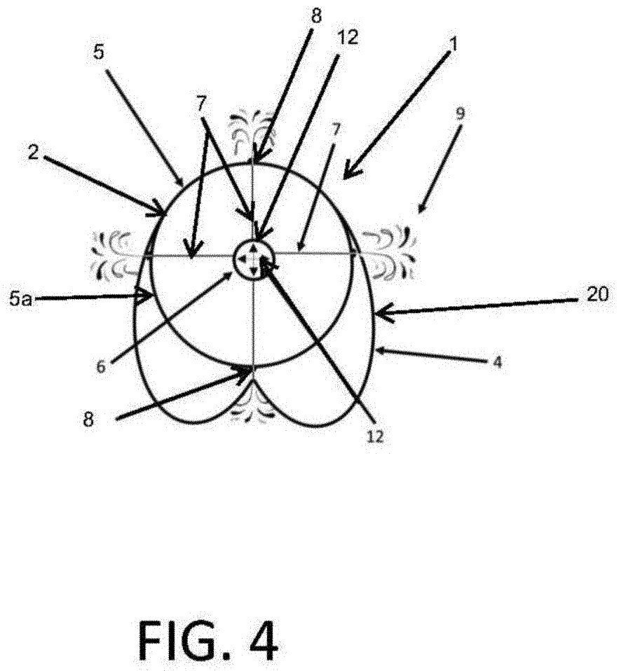

As can be seen from comparison of the different views shown in FIGS. 1-3 and the cross-section end view provided in FIG. 4, the applicator 1 includes five sets of release ports 8, with four sets of release ports positioned along the length of the shaft 5 and another set of release ports 8 positioned along the tip. The release ports 8 positioned along the shaft 5 positioned approximately 90 degrees apart relative to a center of the shaft 5 or the bulk channel 6. That is, four branch channels 7 extend laterally from the bulk channel 6 to the outer surface 5a at approximately perpendicular angles to adjacent branch channels. The branch channels 7 within each set connect to the bulk channel 6 along approximately a same lateral cross-section of the shaft 5. The set of release ports 8 located along the tip 3a include four relatively evenly spaced apart release ports 8. The associated branch channels 4 extend laterally from the bulk channel 6 at forwardly directed angles. In one embodiment, the branch channels 7 are narrower than the bulk channel 6. In one embodiment, the bulk channel 6 comprises an internal shaft through which the composition passes between the reservoir 4 and release ports 8. Such a bulk channel 6 may be flexible or rigid. As described in more detail below, different arrangements of channels 6, 7 and/or release ports 8 may be used.

In various embodiments, the composition 10 may include a lubricant, medicated composition, or cleaning composition. The composition 10 may be an aqueous or non-aqueous liquid, solution, suspension, cream, gel, ointment, lotion, emulsion, oil, or other suitable composition for dispensing by the dispenser device 1.

The delivery device 1 may be designed to deliver medication, lubricant, and/or other compositions 10 into the vagina or anus in a uniform amount depending on how many times or a length of time a user squeezes the reservoir and or moves the applicator 2 into and out from the cavity.

In use, a supply of the composition 10 may be supplied to the applicator 2. For example, the composition 10 may be supplied into the reservoir 4 and thereafter flowed into 11 and through 12 the channels 6, 7 to the release ports 8 for release 9. In the illustrated embodiment, the composition 10 is positioned within the reservoir 4 and may be urged to flow 11 from the reservoir 4 into the bulk channel 6. Flow 11 of the composition 10 into the bulk channel may be driven by manual or mechanically driven or assisted squeezing of the reservoir 4, which may be formed of an elastomeric material, e.g., a rubber-like bulb. In the illustrated embodiment, the handle 20 is provided along the reservoir 4, allowing the user to easily squeeze the reservoir 4 during use. In some embodiments, the dispenser device may be equipped with an actuator or pump configured to drive or pump the composition 10 along the channels 6, 7 and out from the release ports 8. In one embodiment, the reservoir 4 includes a flexible bag that attaches to the bulk channel and squeezing the handle 20 squeezes the bag to flow 11, 12 the composition 10 into and along the channels 6, 7. In one embodiment, the reservoir 4 includes a housing within which the composition is placed. A pump or actuator may then be operable by the user via manipulation of a button, knob, or other user interface along the handle 20 to flow 11, 12 the composition 10 into and along the channels 6, 7.

In an above or another embodiment, the release ports 8 include a one way valve or the like that prevents backflow of the composition 10 or other fluid into the channels 6, 7. The shaft 5 or outer surface 5a thereof may be formed of a flexible and/or elastomeric material than flexes or bends in response to a user manipulating the shaft, e.g., moving the shaft into and out of an orifice, such that the flexes and/or bends reduce the available volume within the channels 6, 7 to eject the composition 10 from the release ports 8 and thereafter replace the composition 10 within the channels 6, 7 when the available space increases. The reservoir 4 may otherwise be sealed to the external environment from entering the reservoir 4 to equalized pressure within the channels 6, 7 that would inhibit the composition 10 from being pulled from the reservoir 4 into the channels.

The delivery device 1 may be provided in a variety of shapes and sizes. As shown, the delivery device 1 is primarily shaped for use in body orifices, to expel and dispense a composition 10, such as a medication, lubricant, or cleaner. The applicator 2 may have a phallic shape, for example. This dispensing device 1 may be manufactured in various sizes, e.g., the length of the shaft 5 may be shortened or lengthened depending on its intended use. The girth of the shaft 5 can be made narrow or wider and the reservoir 4 made smaller or larger. In one embodiment the device 1 is wide at the tip 3a and narrower toward the base 3b and/or reservoir 4. For example, the tip 3a may include an outer perimeter larger than that of an adjacent length of the shaft 5. The handle 20 may be an ergonomic/extended handle from the applicator 2 to allow potential hands-free application where a user's fingers do not have to enter body cavities/orifices during use.

The dispensing device may be constructed from a variety of materials such as, but not limited, to rubber, synthetic rubbers, plastics, silicones, silicone rubbers, glass, wood, metals, metal alloys, ceramics, hemp, biodegradable components, and/or hybrid materials such as borosilicate glass, e.g., PYREX®. Depending on the material of the dispenser device 1, the dispenser device 1 may be disposable, biodegradable, and/or capable of disintegrating within the body. In some embodiments, the dispensing device may be inflatable, expandable, or augmentable through attachments. In one embodiment, the shaft 5 may be configured to vibrate. For example, a button, switch, knob, or other user interface along the handle 20 or elsewhere may be operable to initiate a motor, solenoid, or electromagnetic coil to generate vibrations along the shaft 5.

In use, the composition 10 may be positioned within the reservoir 4. For example, the composition may be inserted, filled, or stored within the reservoir 4. The reservoir 4 may be sized to hold a varied amount of composition 10 for dispensing. The reservoir 4 may fluidically couple to the bulk channel 6 and may be integrated or separate from the handle 20. For example, the reservoir 4 may include a bag or housing that fluidically couples to the bulk channel 6.

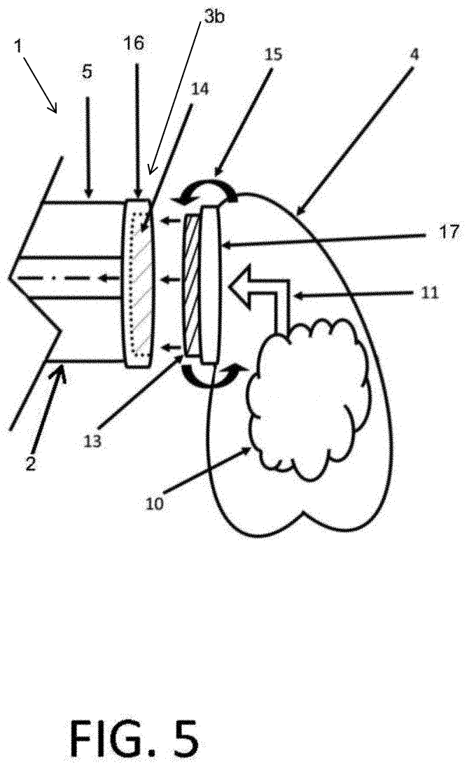

The reservoir 4 may physically connect to the bulk channel 6, which may include selective connection, or may position within the handle 20, which upon connection with the applicator 2, positions the reservoir 4 in fluid communication with the bulk channel 6. In the illustrated embodiment, the reservoir 4 is integrated with the handle 4, and connecting the reservoir 4 to the applicator 2 attaches the handle 20 to the applicator 2 and positions the reservoir 4 in fluid communication with the bulk channel 6. The applicator 2 and reservoir 4 each include a fitting 16, 17 configured to selectively couple and decouple. Various fitting configurations may be used, such as clips, clamps, compression fitments, snap fitments, threaded connections, combinations thereof, or other suitable fitment configurations. As best shown in the partial view of FIG. 5, the applicator fitting 16 is positioned along the base 3b and includes a grooved female connector 14 and the reservoir fitting 17 includes a grooved male connector 13. The male connector 13 may be positioned within the female connector 14 and rotated, e.g., clockwise about 180 degrees, as indicated by arrows 15 to lock the fittings 16, 17 together. It is to be appreciated that the connectors 13, 14 may be reversed as well as the direction of rotation to lock/unlock or couple/decouple the fittings 16, 17. When connected, the reservoir 4 may seal relative to the bulk channel 6 such that exterior fluid may not enter the bulk channel 6 through the seal or the reservoir 4.

While the dispensing device has been described with reference to the drawings, various other configurations and embodiments are contemplated. For example, while a centrally located bulk channel 6 is shown extending down the shaft 5 and connecting to a plurality of branch channels 7, in some embodiments, multiple bulk channels 6 are included. Each of the bulk channels 6 may connect to two or more branch channels 7. Each of the branch channels 7 may connect to a release port 8. Thus, one or more bulk channels 7 may extend down the shaft 5 and directly or indirectly connect, e.g., via a branch channel 6, to one or more release ports 8. In one example, multiple bulk channels 7 extend down the shaft 5, adjacent to the outer surface 5a, and connect directly to release ports 8. In another example, a bulk channel 6 or other channel is provided circumferentially or otherwise around the shaft 5, just below the outer surface 5a. In one configuration, the outer surface 5a may form a sleeve around an inner surface of the shaft 5 between which the bulk channel 6 is formed. In a further example, the outer surface 5a may be formed of flexible and/or elastomeric material such that movement of the applicator 2 into and out of an orifice assists in ejecting the composition 10 from the bulk channel 6 between the inner surface and outer surface 5a of the shaft 5.

In a further or another embodiment, a bulk or branch channel 6, 7 connects to a chamber that connects to multiple branch channels 7 that connect directly or indirectly to one or more release ports 8. While bulk channels 6 and branch channels 7 are shown in the drawings, in various embodiments, each release port 8 connects directly to a bulk channel. In another or further example, branch channels 7 may branch off into one or more additional branch channels 7 that may each connect to one or more release ports 8. In some embodiments, one or more branch channels 7 extend from the bulk channel 6 at non-lateral angles, e.g., angled toward the tip 3a or base 3b.

The illustrated embodiments depict five sets of relief ports 8 relatively evenly spaced along the length of the shaft 5, with each set including four relatively evenly spaced apart release ports 8 positioned around a perimeter of the shaft 5 at approximately 90 degree intervals. However, additional or fewer sets of release ports 8, with each set including additional or fewer release ports 8 may also be used. Sets of release ports 8 may include a same or different number of release ports 8. Release ports 8 within a set of release ports 8 may be located along a cross-section perpendicular to the length of the shaft 5 or one or more release ports 8 may be positioned offset from such a cross-section. Various arrangements of branch channels 7, whether or not associated with a set of release ports 8 may be used. For example, one or more branch channels 7 may extend from the bulk channel 6 from a common junction with one or more other branch channels 7 or individually.

In various embodiments, different numbers and/or locations of sets of release ports 8, release ports 8 within a set of release ports 8, or release ports 8 thereof may be used to provide more targeted administration. Notably, the present disclosure is not limited to release ports 8 associated with sets of release ports 8, as some embodiments may include release port 8 configurations that do not lend themselves to organization suitable to be described as within sets. Release ports 8 may be numbered and evenly positioned to provide even dispensing of composition along the length of the shaft 5. However, the number of release ports 8 along sides (upper, lower, right, left, and in-between) of the outer surface 5a of the shaft 5 may be varied to suit a desired application. For example, a greater number of release ports 8 may be provided along right and left sides. In some embodiments, the size and/or area of the release ports 8 may vary to provide varied volume of dispensing. For example, the area of release ports 8 may reduce from the tip 3a to the base 3b. In some embodiments, additional release ports 8 may be provided along the tip 3a. In one example, one or more release ports 8 are provided to deliver composition 10 from the tip 3a approximately parallel to the longitudinal axis of the shaft 5.

In various embodiments, release ports 8 may be flush with the outer surface 5a. In other embodiments, all or a portion of the release ports 8 may comprise exterior protrusions or nipples that protrude from the outer surface 5a.

The shaft 5 depicted in FIGS. 1-5 is cylindrical and has an arcuate lateral cross-section. However, some embodiments may include other cross-section shapes such as geometric, non-geometric, or free form shapes. In one embodiment, the applicator 2 includes one or more additional shafts (not shown) that extend parallel or non-parallel to the shaft 5. The additional shafts 5 may be longer, shorter, and/or have greater or lesser girth than shaft 5. The outer surface 5a of the shaft 5 may be smooth or may include one or more elevated or recessed surface contours. In some embodiments, raised surface contours may be used to stimulate sensitivity of or secretions from mucosal tissue lining body orifices and/or to enhance absorption of composition 10 during use.

In one embodiment, the dispensing device 1 may include a supply port (not shown) for coupling to a supply of a composition 10. In one example, the supply port may be in addition to or instead of the reservoir 4 and may include a connection for fluidically coupling to a tube or other reservoir or container containing or providing a transport path for the composition 10. For instance, a separate or axillary applicator channel or channels may also be used, which may be similar to that described herein with respect to the bulk channel 6 and branch channel 7. In various embodiments, the supply port may feed directly into the reservoir 4 or bulk channel 6 for mixing with or replacing the composition 10 in the reservoir 4 or may be fed into an auxiliary or separate reservoir 4 or applicator channel for dispensing from separate release ports 8. For example, the composition 10 supplied into the supply port may thereafter mix with the same or another composition 10 within the reservoir 4, bulk channel 6, branch channel 7, or intermediate chamber fluidically coupled or coupleable thereto.

In one example, a cleaning composition may be dispensed from the applicator 2 and thereafter a user may switch to dispensing a rinsing solution to provide a rinsing solution from the same or a different release ports 8. The rinsing solution may be supplied through a supply port or by coupling of a reservoir 4 including the rinsing solution to the applicator 2. The rinsing solution may be water, for example, to flush the cleaning composition and debris.

In an above or another embodiment, the reservoir 4 includes multiple chambers selectable by a user via a toggle button, switch, knob, or the like, which may be provided on a user interface positioned along the handle 20 or elsewhere. For example, toggling may operate a valve to one or more chambers to supply the contents of the one or more chambers into the applicator 2. Toggling may also be provided to allow a user to selectively open and close valves to selected chambers, e.g., for sequential administration of different compositions 10.

According to various dispensing methods, it may be practical to leave the dispensing device 1 within the body of the subject or user indefinitely, connected or disconnected to external tubing, e.g., via a supply port. Leaving the dispensing device 1 within the body may be more practical or comfortable if the dispensing device 1, such as the applicator 2 has a small size. The lubrication provided by dispensing of a lubricant may therefore last indefinitely, which may be based on reservoir 4 capacity. In some such embodiments, the handle 20 may be excluded or not connected. Similarly, the reservoir 4 may be reduced in size or connected to the bulk channel 6 by a supply port, such via a hose, to allow the reservoir 4 to be worn by the user or otherwise located separate from the applicator 2. In one example, the reservoir 4 may be configured to position within the shaft 5 or external to the base 3b and have a thin or unobtrusive formfactor. If the dispenser device 1 is not suitable to remain in the body permanently, it may still be suitable for prolonged insertion similar to an anal plug, vaginal plug, or even menstrual cup. In some embodiments, the dispenser device 1 may be configured for controlled time release dispensing of compositions 10. For example, a pump, motor, actuator or other device configured to cause flow of the composition through the applicator 2 and from the release ports 8 may be operable by a control system programmed to control rate of dispensing, times and/or volume of dispensing. In one configuration, a user interface is provided to allow a user to select desired dispensing programs.

The dispenser device 1 may be supplied with one or more compositions 10 to be dispensed contained in the reservoir 4. In some embodiments, the dispenser device 1 may be supplied with one or more compositions 10 that the user may supply into the reservoir 4 prior to dispensing the composition 10. The dispenser device 1 may also be supplied separate from compositions 10 and a user may identify and/or choose which compositions 10 to be supplied into the dispenser device 1 for dispensing. In some embodiments, a plurality of reservoirs 4 prefilled with various compositions 10 may be available for purchase. Prior to dispensing, the user may couple a selected reservoir 4 to the applicator 2.

As introduced above, the dispenser device 1 may be used to dispense various compositions 10. The composition 10 may be or include, for example, a lubricant, medication, and/or cleaning composition. Such compositions 10 or other compositions 10 that may be dispensed by the dispenser device 1 may be in the form of a liquid, cream or a solidified oily blend, emulsion, or slurry. In other embodiments the composition 10 exhibits a more viscous liquid and can be produced with a variety of viscosities that are compatible with and can pass through the channels 6, 7 and release ports 4 of the dispenser device 1. For example, certain varieties of the composition 10 may have a viscosity with the consistency of water, oil, or a lotion. Some compositions 10 may be in the form of gels.

The dispensing device 1 may be used in a method to provide a composition 10 for providing medication, lubrication, cleansing composition to a subject via the anal or vaginal orifice. The subject may be a mammal, such as a human. Additionally or alternatively, dispensing the composition 10 may provide pain relief, moisturization, shine, lubrication, inflammation reduction, sexual stimulation, psychoactive stimulation, aromatics, and cosmetic benefits. The composition 10 may include penetrant enhancers such as oils, essential oils, urea, or dimethyl sulfoxide (DMSO).

In some embodiments, the composition 10 may be or include a lubricant and/or medicated composition, and the dispenser device 1 comprises a lubricant and/or medicine dispersion device for dispersing the composition 10. For example, the dispenser device 1 may operate as a lubricant and/or medicine applicator tool to expel, disperse, squirt, or similarly dispense a sexual lubricant, massage lubrication, and/or medication within an anal or vaginal orifice. This delivery technique may treat the vaginal canal or anus with beneficial effects similar to suppositories, with the added benefits of massaging movements. The dispenser device 1 may similarly be used to deliver such compositions 10 or other compositions 10 onto other body surfaces or into other body orifices. In another application, the dispenser device 1 may be used to deliver such compositions 10 or other compositions 10 into or onto equipment like sex implements, toys, or aids, e.g., dildos, sex dolls, condoms, plugs, or spinning/rubbing devices. The composition 10 may include a lubricant that may be applied, for example, onto parts or areas that provide mechanical friction. The lubricant may be a non-toxic lubricant.

Compositions 10 comprising cleaner or cleaning composition may be selected to clean a body surface, orifice, or tissue to which the composition 10 is administered. For example, the composition may include a soap, antiseptic, alcohol, iodine, or peroxides, such as hydrogen peroxide. In some embodiments, the composition includes a spermicide. In some applications, a cleaning composition is configured to clean an artificial surface. For example, the composition 10 may include a solution including soap, alcohol, acid, sodium hydroxide, sodium hypochlorite, hydrogen peroxide, disinfectants, biocides, or combinations thereof

Compositions 10 comprising medicated compositions may be administered to a subject for systemic or local action. Medicated compositions may be in the form of a liquid solution or suspension, emulsion, gel, lotion, cream or a solidified oily blend. For example, medications delivered with the dispenser device may be delivered into the entire body, such as transmucosal, through mucosal tissue absorption by way of the vaginal cavity or anus or may be delivered into the vaginal or anus area to treat local symptoms of pain, infection, cramping, or dryness locally at or in the specific area of administration.

The dispenser device 1 may be utilized to deliver compositions 10 providing localized effects to the vagina or the rectum and colon. Once within the vagina or rectum, the composition 10 is quickly delivered directly to the bloodstream through the vaginal or rectal walls, effectively reaching a near-one hundred percent absorption rate. The composition 10 may help tissue repair, prolapsed hemorrhoids, pH balance, muscle recovery, athletic performance, mental relaxation, menstrual cramp discomfort.

Medicated compositions may be used to treat pain, stiffness, muscle spasms, organ transplant rejection, atopic dermatitis, and symptoms of epilepsy. In one embodiment, the composition 10 comprises a topical cream or lotion designed primarily for health, rejuvenation, and symptom relief across human flesh, the circulatory system, and the nervous system. In another embodiment, the composition 10 comprises a gel-like skin treatment serum designed to preface other topical treatments.

In some embodiments, medicated compositions may include or may function as a potential alternative to synthetic pharmaceuticals such as: clotrimazole, miconazole, carisoprodol, prasterone, diazepam, bisacodyl, acetaminophen, tacrolimus, immunosuppressive drugs, alprazolam, or combination thereof. In various embodiments, the composition 10 includes one or more appetite stimulants (orexigenics), athletic performance supplements, menstrual symptom relief cream, tinctures as antifungals, antifungal creams, vaginal suppositories, benzoyl peroxide, resorcinol, salicylic acid, and sulfur, anti-scarring topicals including, e.g., arbutin glycoside and/or kojic acid, nerve depressants, saline wash with CBD for reduced inflammation, epilepsy and seizure treatments, CBD diluted with saline and hyaluronic acid, probiotics, massage creams, massage oils, Witch Hazel with optional THC and CBD, endocannabinoid infusions, soaps, sleep aids, sedatives, sunscreens, numbing creams, weight-loss substances, anti-inflammatory blends, anti-aging creams, topical creams that reverse aging effects, abolish potential hazards, or prevent and/or reduce damage from sources that inhibit cell regeneration, growth, repair, and cell turnover.

The composition 10 may include one or more endocannabinoids or one or more exogenous cannabinoids. In some embodiments, endocannabinoids may be selected from lysophosphatidylinositol, 2-arachidonoylglycerol, 2-arachidonyl glyceryl ether, N-arachidonoyl dopamine, anandamide, virodhamine, and combinations thereof In one example, the composition 10 includes at least on exogenous cannabinoid selected from THC (Δ9-tetrahydrocannabinol), CBD (Cannabidiol), CBC (Cannabichromene), CBGV (Cannabigerivarin), THCV (Tetrahydrocannabivarin), CBDV (Cannabidivarin), CBCV (Cannabichromevarin), CBN (Cannabinol), and CBG (Cannabigerol).

In various embodiments, the composition 10 comprises a sex lubricant for the vagina and features combinations of THC, CBD, terpenes, flavors, fragrances, and oily or watery compounds. In another embodiment, the composition 10 comprises a sex lubricant for the anus and features combinations of THC, CBD, terpenes, flavors, fragrances, and oily or watery compounds. In another or a further embodiment, a medicated sex lubricant includes a contraceptive. In another embodiment, a sex lube for sex tools and toys features combinations of THC, CBD, terpenes, oily compounds, and watery compounds.

A method of treating appetite and digestion, metabolism, chronic pain, inflammation and other immune system responses, mood, learning and memory, motor control, sleep, cardiovascular system function, muscle formation, bone remodeling and growth, liver function, reproductive system function, skin and/or nerve function may include administering a composition 10 to a subject that includes one or more endocannabinoids and/or exogenous cannabinoid, such as those identified herein, to the vaginal or anal orifice using the dispenser device 1. Additionally or alternatively, the composition 10 may include one or more flavonoids and/or terpenes. In one embodiment, the composition 10 includes terpenes, THC, THCv, CBD, Epigallocatechin-3-O-gallate (EGCG), and, optionally, one or more anti-inflammatories, nutraceuticals, appetite suppressants, or ayurvedic compounds.

Administration of a composition 10 including one or more endocannabinoids and/or exogenous cannabinoids, such as those identified herein, using the dispenser device 1 may be used to treat mood swings, anxiety, inflammation, and sleep disorders, relieve tension and stress in subjects suffering from mental trauma or PTSD, fibromyalgia, pain, depression, anorexia, Irritable Bowel Syndrome, migraines, Muscular Sclerosis, neuropathy, Huntington's, Parkinson's, or autism. Additionally or alternatively, the composition 10 may include one or more flavonoids and/or terpenes. In one embodiment, the composition 10 includes terpenes, THC, THCv, CBD, Epigallocatechin-3-O-gallate (EGCG), and, optionally, one or more anti-inflammatories, nutraceuticals, appetite suppressants, or ayurvedic compounds.

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth in this specification. Such embodiments may be obtained, for example, by combining, modifying, or reorganizing any of the disclosed steps, components, elements, features, aspects, characteristics, limitations, and the like, of the various non-limiting and non-exhaustive embodiments described in this specification.

The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” means one or more components, and thus, possibly, more than one component is contemplated and may be employed or used in an application of the described embodiments. Further, the use of a singular noun includes the plural, and the use of a plural noun includes the singular, unless the context of the usage requires otherwise. Additionally, the grammatical conjunctions “and” and “or” are used herein according to accepted usage. By way of example, “x and y” refers to “x” and “y”. On the other hand, “x or y” refers to “x”, “y”, or both “x” and “y”, whereas “either x or y” refers to exclusivity.

The present disclosure may be embodied in other forms without departing from the spirit or essential attributes thereof and, accordingly, reference should be had to the following claims rather than the foregoing specification as indicating the scope of the invention. Further, the illustrations of arrangements described herein are intended to provide a general understanding of the various embodiments, and they are not intended to serve as a complete description. Many other arrangements will be apparent to those of skill in the art upon reviewing the above description. Other arrangements may be utilized and derived therefrom, such that logical substitutions and changes may be made without departing from the scope of this disclosure.

1. A dispenser device for delivering a composition into an anal or vaginal orifice, the device comprising:
an applicator comprising:
a shaft that extends from a tip to a base and including an outer surface;
a plurality of release ports positioned along the outer surface of the shaft; and
one or more channels extending to and in fluid communication with the plurality of release ports, wherein the one or more channels extend through the shaft from the plurality of release ports toward the base of the shaft.

2. The device of claim 1, wherein the device further comprises a reservoir configured to contain a composition to be dispensed from the device.

3. The device of claim 2, wherein the reservoir is configured to couple to the applicator, and wherein the reservoir is in fluid communication with the one or more channels when coupled to the applicator.

4. The device of claim 3, wherein the reservoir and applicator each include a fitting configured to engage and couple the reservoir to the applicator.

5. The device of claim 4, wherein the device further comprises a handle, and wherein the handle is integrated with the reservoir.

6. The device of claim 4, wherein the reservoir comprises a flexible and/or elastomeric material configured to be squeezed by a user to urge a composition within the reservoir to flow from the reservoir and through the one or more channels.

7. The device of claim 4, wherein the applicator fitting is positioned adjacent to the base of the shaft and includes a grooved female connector, wherein the reservoir fitting includes a grooved male connector configured to be positioned within the female connector and rotated to couple the applicator and the reservoir.

8. The device of claim 1, wherein the one or more channels comprise a bulk channel that extends from the base of the shaft toward the tip of the shaft.

9. The device of claim 8, wherein the one or more channels further comprise a plurality of branch channels, and wherein each of the branch channels extend between the bulk channel and one of the release ports.

10. The device of claim 1, wherein the plurality of release ports are located along the tip and sides of the shaft.

11. The device of claim 1, wherein the plurality of release ports include protrusions that protrude from adjacent portions of the outer surface of the shaft.

12. A method of administering a composition to a subject, the method comprising:
inserting a dispensing device within an anal or vaginal orifice, wherein the dispensing device comprises:
an applicator comprising:
a shaft that extends from a tip to a base and including an outer surface,
a plurality of release ports positioned along the outer surface of the shaft, and
one or more channels extending to and in fluid communication with the plurality of release ports, wherein the one or more channels extend through the shaft from the plurality of release ports toward the base of the shaft, and
a reservoir configured to contain the composition to be dispensed from the device, wherein the reservoir is in fluid communication with the one or more channels; and
ejecting the composition from the plurality of release ports within the anal or vaginal orifice.

13. The method of claim 12, wherein the reservoir comprises a flexible and/or elastomeric material, and wherein ejecting the composition comprises squeezing the reservoir to urge the composition within the reservoir to flow from the reservoir and through the one or more channels and out of the plurality of release ports.

14. The method of claim 12, wherein the reservoir and applicator each include a fitting configured to engage and couple the reservoir to the applicator.

15. The method of claim 12, wherein the one or more channels comprise a bulk channel that extends from the base of the shaft toward the tip of the shaft.

16. The method of claim 15, wherein the one or more channels further comprise a plurality of branch channels, and wherein each of the branch channels extend between the bulk channel and one of the release ports.

17. The method of claim 12, wherein the plurality of release ports are located along the tip and sides of the shaft.

18. The method of claim 12, wherein the plurality of release ports include protrusions that protrude from adjacent portions of the outer surface of the shaft.

19. The method of claim 12, wherein the composition comprises one or more cannabinoids.

20. The method of claim 19, wherein the composition is a liquid, cream, or an oily blend.

21. The method of claim 14, wherein the applicator fitting is positioned adjacent to the base of the shaft and includes a grooved female connector, and wherein the reservoir fitting includes a grooved male connector configured to be positioned within the female connector and rotated to couple the applicator and the reservoir.
